(19) United States
(12) Patent Application Publication
(10) Pub. No.: US 2017/0105637 A1
(43) Pub. Date: Apr. 20, 2017

(54) CONTINUOUS NON-INVASIVE BLOOD PRESSURE MONITOR

(71) Applicants: Tony Joseph Akl, Woburn, MA (US); Paul V. Errico, Andover, MA (US)

(72) Inventors: Tony Joseph Akl, Woburn, MA (US); Paul V. Errico, Andover, MA (US)

(21) Appl. No.: 14/885,672

(22) Filed: Oct. 16, 2015

(51) Int. Cl. A61B 5/021; A61B 5/00

(52) U.S. Cl. CPC A61B 5/02125; A61B 5/02141; A61B 5/725; A61B 2562/0214; A61B 2562/0238

(57) ABSTRACT

A blood pressure monitoring system is provided comprising: an impedance monitoring circuit configured to detect an occurrence of a blood pulse wave ejection from the heart; a blood pulse detection circuit detect to an arrival of the blood pulse wave at a body site peripheral to the heart; and a processor configured to compute pulse transit time (PTT) based at least in part upon a difference in a time of occurrence of the blood pulse wave ejection from the heart first signal and a time of occurrence of the arrival of the blood pulse wave at a body site peripheral to the heart.

### BACKGROUND

Blood pressure (BP) is the pressure exerted by the circulating blood on the blood vessel walls of the circulatory system. Each beat of the heart pumps a blood pressure pulse wave out from the heart into the blood vessels. The blood pressure pulse waves propagate throughout the circulatory system causing pulses of blood to course through the blood vessels and to reach peripheral parts of the body. Each blood pressure pulse wave exerts a pressure wave upon the blood vessel walls as it surges through. The pulse wave velocity (PWV) is the speed of a blood pressure pulse propagating along the blood vessel walls. The PWV can be calculated based upon pulse transit time (PTT), which is the time required for a blood pressure pulse wave to propagate on the same cardiac cycle from a blood vessel site closer to the heart to a blood vessel site more distant from the heart. The relationship between PWV and PTT can be expressed as follows,

$$P W V = \frac{x}{P T T} , \qquad ( 1 )$$

The parameter x represents a distance between two blood vessel sites between which a blood pressure pulse wave propagates, such as a distance between the heart and a specified blood vessel site that is peripheral to the heart. PTT has been shown to be quasi-linear to low blood pressure values, but to increase exponentially at higher pressures.

The velocity of a longitudinal blood pressure wave is related to arterial pressure and the intrinsic elastic properties of the arterial wall. The Moens-Korteweg equation expresses the relationship as follows,

$$P W V = \sqrt{\frac{g E h}{ρ d}} \qquad ( 2 )$$

The parameter g is the acceleration of gravity, E is the elastic modulus of the blood vessel walls, h is the thickness of the vessel walls, p is the density of blood, and d is the blood vessel diameter. See, Gesche, H. et al., Continuous blood pressure measurement by using the pulse transit time: comparison to a cuff-based method, Eur. J. Appl. Physiol., April 2011.

The elastic modulus of the blood vessels has an exponential relationship to BP:

E=E₀e^{γ·BP} (3)

The parameter E0 is the elastic modulus at zero pressure and γ is a coefficient of vascular characterization.

The following relationship between change in blood pressure and change in PTT has been determined based upon the above relationships:

$$dB P = \frac{2}{γ T} d P T T \qquad ( 4 )$$

This relationship signifies that if the blood vessel elasticity remains unchanged, then changes in BP are proportional to changes in PTT. Thus the changes in BP can be identified by identifying changes in PTT. See, Sheng, H. et al., A wireless wearable body sensor network for continuous noninvasive blood pressure monitoring using multiple parameters, Recent Researches in Circuits, Systems, Communications and Computers, ISBN: 978-1-61804-056-5, pages 308-314, at 310.

Various techniques have been used to measure PTT. For example, Chen et al. Continuous and noninvasive blood pressure measurement: a novel modeling methodology of the relationship between blood pressure and pulse wave velocity, Ann. Biomed. Eng, Vol. 40, No. 4, April 2012 pages 871-882, teach photoplethysmography (PPG) measurements at ear and toe to determine PTT. Myint, C Z. et al., Blood Pressure Measurement from Photo-plethysmography to Pulse Transit Time, 2014 IEEE Conference on Biomedical Engineering and Sciences, 8-10 Dec. 2014, Miri, Sarawak, Malaysia, pages 496-501, teach PPG measurements at wrist and finger to determine PTT. Also, for example, Sheng et al. teach a combination of an ECG signal and a PPG signal to determine PTT.

Moreover, various mathematical relationships have been found between BP and PTT and PWV. For example, Chen et al. proposed the following relationship,

$$B P = b {e}^{- \frac{k}{P W V}} \qquad ( 5 )$$

The parameters k and b are calibration parameters that depend on the age and gender of the user.

Also, for example, Shriram et al. proposed the following relationship,

$$B P = \frac{1}{α} [ ln ( \frac{{x}^{2} r B P}{{E}_{0} h} ) - 2 ln ( P T T ) ] \qquad ( 6 )$$

The parameter r is the inner radius of the artery, and the other parameters of equation (6) are the same as corresponding parameters defined above. See, Shriram, R. et al., Continuous cuffless BP monitoring based on PTT, Bioinformatics and Biomedical Technology (ICBBT), 2010 International Conference, Chengdu, China, April 2010.

### SUMMARY

In one aspect, a blood pressure monitoring system is provided that includes an impedance measurement circuit configured to detect a change in impedance indicative of an occurrence of a blood pulse wave ejection from the heart. The system includes a blood pulse detection sensor to detect arrival of the blood pulse wave at a body site peripheral to the heart. A non-transitory computer readable storage device is provided that includes computer program code to configure a processor to compute pulse transit time (PTT) based at least in part upon a difference in a time of occurrence of the blood pulse wave ejection from the heart first signal and a time of occurrence of the arrival of the blood pulse wave at a body site peripheral to the heart.

In another aspect, a method is provided to monitor blood pressure. The method includes monitoring electrical impedance at first and second body skin tissue sites and monitoring blood pressure at a third skin tissue site. A PTT is determined based at least in part upon a difference in a time of occurrence of a monitored change in impedance at the first skin tissue site and a monitored change in blood pressure at the second skin tissue site.

### BRIEF DESCRIPTION OF THE DRAWINGS

FIG. 1 is an illustrative drawing representing a human circulatory system and showing illustrative blood pressure pulse waveforms that propagate from the heart throughout the circulatory system.

FIG. 2 is an illustrative an impedance measurement system and a blood pulse detection sensor operatively coupled to detect blood pulses in the human circulatory system, in accordance with some embodiments.

FIG. 3 is an illustrative flow diagram representing a process to measure PTT in accordance with some embodiments.

FIG. 4 is an illustrative drawing showing certain details of the impedance measurement system of FIG. 2 in accordance with some embodiments.

FIG. 5 is an illustrative graph showing an experimental result of impedance measurement across the chest cavity versus time for two different respiration rates.

FIG. 6 is an illustrative block diagram showing certain other details of the impedance measurement system of FIG. 2 in accordance with some embodiments.

FIG. 7 is an illustrative schematic block diagram showing certain details of a blood dynamics sensor in accordance with some embodiments.

FIG. 8 is an illustrative drawing showing a computer system coupled to process PTT related information in accordance with some embodiments.

FIG. 9 is an illustrative flow diagram representing configuration of the processor of FIG. 8 to determine PTT values in accordance with some embodiments.

### DESCRIPTION OF EMBODIMENTS

The following description is presented to enable any person skilled in the art to create and use a continuous non-invasive blood pressure monitor. Various modifications to the embodiments will be readily apparent to those skilled in the art, and the generic principles defined herein may be applied to other embodiments and applications without departing from the spirit and scope of the invention. Moreover, in the following description, numerous details are set forth for the purpose of explanation. However, one of ordinary skill in the art will realize that the invention might be practiced without the use of these specific details. In other instances, well-known processes are shown in block diagram form in order not to obscure the description of the invention with unnecessary detail. Identical reference numerals may be used to represent different views of the same or similar item in different drawings. Flow diagrams in drawings referenced below are used to represent processes. Thus, the present invention is not intended to be limited to the embodiments shown, but is to be accorded the widest scope consistent with the principles and features disclosed herein.

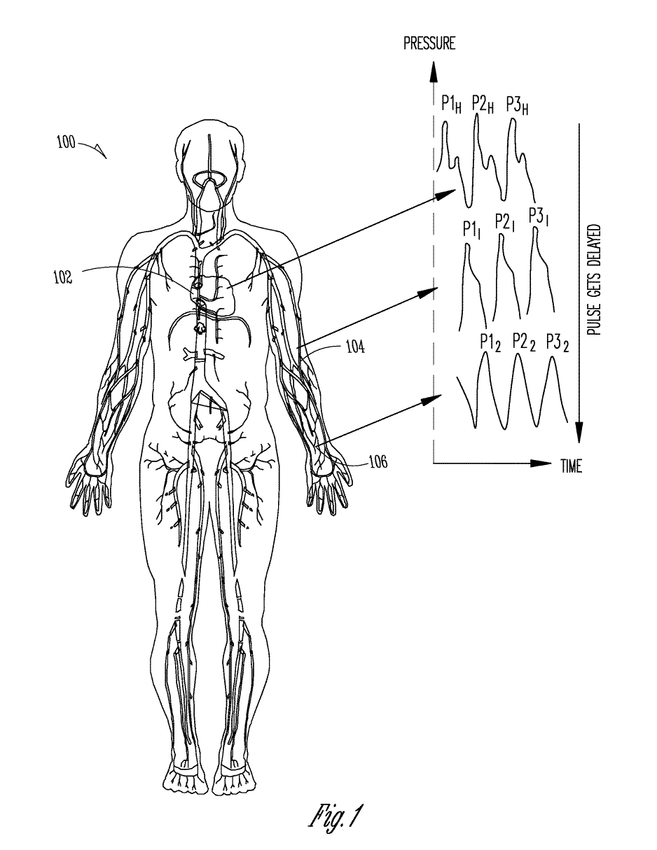

FIG. 1 is an illustrative drawing representing a human circulatory system 100 showing illustrative blood pressure pulse waveforms that propagate from the heart 102 throughout the circulatory system. A sequence of blood pulse waveforms P1, P2, P3, each characterized by a an increase in blood pressure ΔPᵢ upon the arrival of a blood pressure wave, are shown as they propagate from the heart 102 to a first blood vessel site 104 and then to a second blood vessel site 106. A first pulse P1 is ejected followed in sequence by a second pulse P2 and a third pulse P3. The waveform P1_{H} represents the first pulse at the heart site 102. The waveform P1₁ represents the first pulse at the first peripheral blood vessel site 104. The waveform P1₂ represents the first pulse at the second peripheral blood vessel site 106, which is more distant from the heart 102 than is the first site 104. Similarly, waveforms P2_{H}, P2₁ and P2₂ represent the second pulse at the heart and at the first and second blood vessel sites, respectively. Likewise, the waveforms P3_{H}, P3₁ and P3₂ represent the third pulse at the heart and at the first and second blood vessel sites, respectively. It can be seen that the blood pressure pulse waveforms change as they propagate farther from the heart. The change in the pressure pulses is represented by the changes in the shape of the curves representing the pulses at each site.

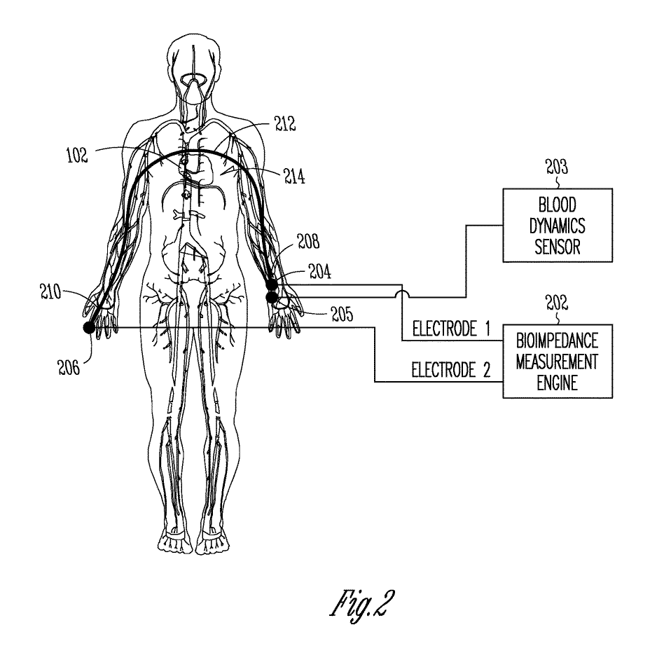

FIG. 2 is an illustrative an impedance measurement circuit 202 and a blood pulse detection sensor 203 operatively coupled to detect occurrence of blood pulses in the human circulatory system 100, in accordance with some embodiments. A change in blood dynamics at a selected location within the circulatory system caused by an arrival of a blood pulse at the location in the circulatory system can involve one or more of a change in blood pressure, a change in blood volume and a change in blood flow, for example, at the selected location The impedance measurement circuit 202 includes first and second electrodes 204, 206 that are suited for monitoring physiological electrical signals produced at body skin tissue sites. In accordance with some embodiments, the first and second electrodes 204, 206 include wet contact, dry contact or noncontactelectrodes. See, Yu Mike Chi et al. Dry-Contact and Noncontact Biopotential Electrodes: Methodological Review, IEEE Reviews in Biomedical Engineering, Vol. 3, pages 106-119, 2010 for a review of the various types of electrodes for biopotential signals. The impedance measurement circuit 202 is used to measure impedance of an electrical signal path between its electrodes. When the electrodes 204, 206 are to monitor physiological electrical signals at different sites on the human body, an impedance measurement system 202 can measure impedance of an electrical signal path comprising body tissue disposed between the different sites. In the illustrative drawing of FIG. 2, the first electrode 204 is electrically coupled to monitor a first site 208 disposed at the left wrist, and the second electrode 206 is electrically coupled to monitor a second site 210 at a finger of the right hand. It can be seen that a body tissue electrical path 212 between the illustrative first site 208 and the illustrative second site 210 crosses the chest cavity 214, which contains the heart 212. Therefore, with the first and second electrodes 204, 206 electrically coupled to monitor the first and second skin sites 208, 210, the impedance measurement circuit 202 can detect changes in impedance of the chest cavity and the heart. The pulse detection sensor 203 detects arrival of a blood pulse wave at a selected site 205 on the body peripheral to the heart 102.

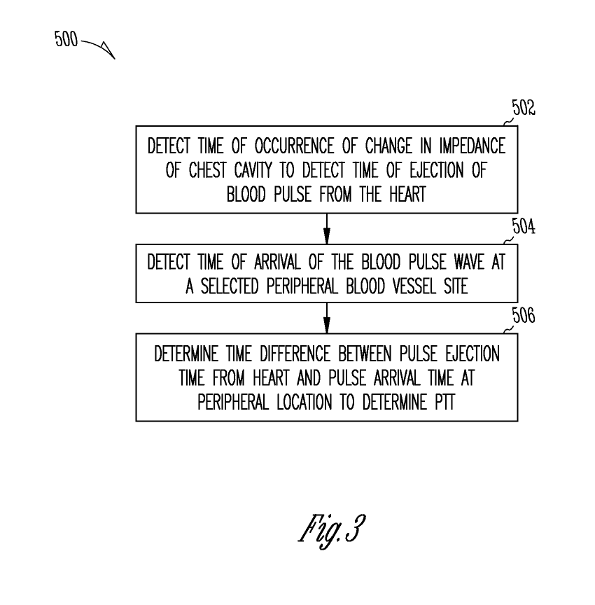

FIG. 3 is an illustrative flow diagram representing a process to measure PTT in accordance with some embodiments. In step 502, a time of occurrence of a change of impedance across the chest cavity 214 is detected to detect the time of ejection of a blood pulse wave from the heart. In some embodiments, the impedance measurement circuit 202 performs this step by monitoring a voltage drop across the chest cavity 214 while conducting a current across the chest cavity, through body tissue. Certain changes in the voltage drop across the chest cavity are indicative of ejection of a blood pulse wave from the heart. In step 504, the time of arrival of the blood pulse wave is detected at a selected peripheral blood vessel site 205. In step 506, PTT is calculated based upon a time difference between the time of blood pulse wave ejection from the heart and the time arrival time of the blood pulse wave at the selected peripheral site 205.

An advantage of using bioimpedance monitoring rather than an ECG monitoring to determine a time of ejection of a blood pulse from the heart is that time of occurrence of ECG signal components such as R-wave and Q-wave often do not coincide precisely with time of occurrence of blood pulse ejection from the heart and the relationship can vary. The difference between the two time points (ECG and blood ejection from the heart) is often referred to as the pre-ejection period. The use of bioimpedance monitoring advantageously does not depend upon identification of ECG waveform components. Rather, bioimpedance monitoring more directly detects time of occurrence of blood pulse ejection based upon changes in impedance that correspond directly to changes in physical shapes of anatomical structures in the course of blood pulse ejection from the heart.

Referring again to FIG. 1 and to waveforms P1, P2 and P3, measurement of PTT involves detecting the arrival time of a pulse at at least two different blood vessel locations that are spaced apart from each other at different distances from the heart 102 within the circulatory system 100. Values measured for PTT may vary from one pulse to the next. Assume, for example, that the first pulse P1_{H} is ejected from the heart at time t₁, and that P1₁ arrives at the first blood vessel site 104 at time t₁+Δ1ₜ₁, and that P1₂ arrives at the second blood vessel site 106 at time t₁+Δ2ₜ₁. Then, for the first pulse, the PTT between the occurrence of Phi at the heart 102 and the occurrence of P1₁ at the first blood vessel site 104 is Δ1ₜ₁, the PTT between the occurrence of P1_{H} at the heart 102 and the occurrence of P1₂ at the second blood vessel site 104 is t₁+Δ2ₜ₁, and the PTT between the occurrence of P1₁ at the first blood vessel site 104 and the occurrence of P1₂ at the second blood vessel site 106 is Δ2ₜ₁−Δ1ₜ₁. Similarly, assuming that the second pulse P2_{H} is ejected from the heart at time t₂, and that P2₁ arrives at the first blood vessel site 104 at time t₂+Δ1ₜ₂, and that P2₂ arrives at the second blood vessel site 106 at time t₂+Δ1ₜ₂. Then, for the second pulse, the PTT between the occurrence of P2_{H} at the heart 102 and the occurrence of P2₂ at the first blood vessel site 104 is Δ1ₜ₂, the PTT between the occurrence of P2₂ at the heart 102 and the occurrence of P2₂ at the second blood vessel site 104 is t₂+Δ1ₜ₂, and the PTT between the occurrence of P2₁ at the first blood vessel site 104 and the occurrence of P2₂ at the second blood vessel site 106 is Δ2ₜ₂−Δ1ₜ₂. Likewise, assuming that the third pulse P3_{H} is ejected from the heart at time t₃, and that P3₁ arrives at the first blood vessel site 104 at time t₃+Δ1ₜ₃, and that P3₂ arrives at the second blood vessel site 106 at time t₃+Δ1ₜ₃. Then, for the third pulse, the PTT between the occurrence of P3_{H} at the heart 102 and the occurrence of P3₁ at the first blood vessel site 104 is Δ1ₜ₃, the PTT between the occurrence of P3_{H} at the heart 102 and the occurrence of P3₂ at the second blood vessel site 104 is t₃+Δ1ₜ₃, and the PTT between the occurrence of P3₁ at the first blood vessel site 104 and the occurrence of P3₂ at the second blood vessel site 106 is Δ2ₜ₃−Δ1ₜ₃.

In accordance with some embodiments, the impedance measurement system 102 can be used to make a first PTT measurement that detects the time of ejection of a blood pulse wave from the heart, and the pulse detection sensor 203 makes a second PTT measurement that detects time of arrival of the same blood pulse wave at a peripheral blood vessel site. Thus, continuing with the above example, for the first pulse, the first PTT would be Δ1ₜ₁, if the first blood vessel site 104 is used for the second PTT measurement and would be Δ2ₜ₁, if the second blood vessel site 106 is used for the second PTT measurement, for example. Similarly, for the second pulse, the second PTT would be Δ1ₜ₂, if the first blood vessel site 104 is used for the second PTT measurement and would be Δ1ₜ₂, if the second blood vessel site 106 is used for the second PTT measurement. Likewise, for the third pulse, the second PTT would be Δ1ₜ₃ or Δ1ₜ₃, depending upon the first or second blood vessel site 104 or 106 is used.

Each waveform represents a heartbeat cycle in which the heart 102 pumps oxygenated blood to the body and deoxygenated blood to the lungs. In a normal healthy person, the resting heartbeat rate is about seventy-two beats per minute. The heart 102 rests momentarily between beats. The heart includes a right atrium in the upper chamber of the right side of the heart. The blood that is returned to the right atrium is deoxygenated (poor in oxygen) and is passed into the right ventricle to be pumped through the pulmonary artery to the lungs for re-oxygenation and removal of carbon dioxide. The heart includes a left atrium that receives newly oxygenated blood from the lungs as well as the pulmonary vein which is passed into the strong left ventricle to be pumped through the aorta to the different organs of the body. The shape and blood fluid content of the heart 102 changes during a heartbeat as the heart pumps blood through its four chambers. The shape and blood fluid content of the heart 102 during a heartbeat is different from its shape and blood fluid content while at rest between beats. The first and second electrodes 202, 204 are disposed to monitor physiological electrical signals at first and second sites 208, 210 on a person's skin such that electrical impedance of an electrical path that includes body tissue disposed between them is different during occurrence of a heartbeat than it is between heartbeats. In accordance with some embodiments, a blood pulse ejection from the heart is defined as an ejection from the left ventricle to the aorta/body. However, other alternative pulse ejection time points can be used for various reasons such as, for example, simplicity or better signal quality.

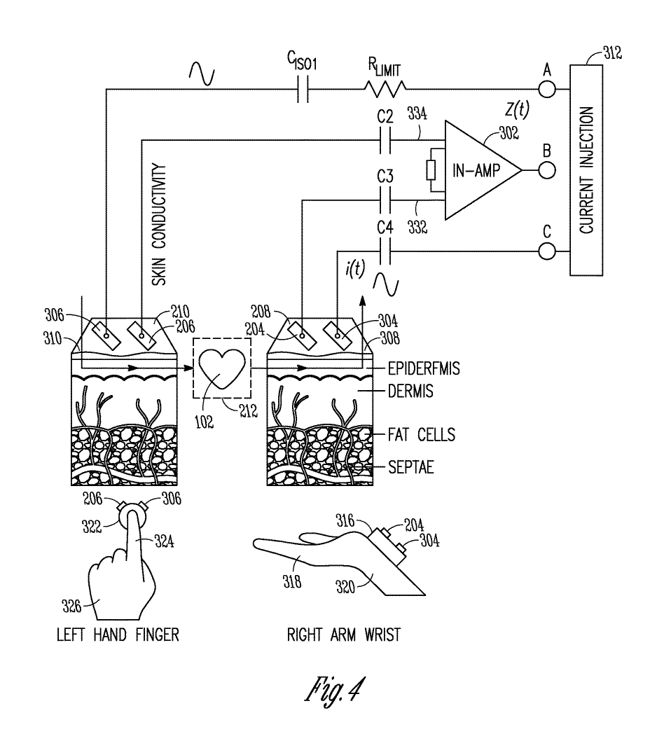

FIG. 4 is an illustrative drawing showing certain details of the impedance sensor circuit 202 of FIG. 2, in accordance with some embodiments. The impedance measurement circuit 202 includes voltage signal measurement circuit 302 and a current injection circuit 303. The voltage signal measurement circuit 302 produces an electrical signal that represents voltage drop across the first and second electrodes 204/304, which represents voltage drop across a body tissue circuit path 212 comprising human body tissue disposed between a first skin surface site 208/308 that is monitored using the first electrode 206/306 and the second skin surface site 310 that is monitored using the second electrode 208/308. The current injection circuit 303 includes a third electrode 304 suited monitoring a third skin site 308 and includes a fourth electrode suited for monitoring a fourth skin site. The current injection circuit 303 is operable to inject current flow through the body tissue circuit path 212, which is disposed between the third and fourth electrodes 308, 310.

The third and fourth electrodes 304, 306 are disposed to physiologically monitor third and fourth body tissue sites 308, 310 on the skin surface so as to define an electrical path between them that is substantially the same as the body tissue electrical path 212 between the first and second electrodes 204, 206. In accordance with some embodiments, the first and third electrodes 204, 304 are disposed to physiologically monitor tissue sites 208, 308 that are close enough together and the second and fourth electrodes 206, 306 are disposed to physiologically monitor tissue sites 210, 310 that also are close enough together, such that a voltage difference between the first and second electrodes 204, 206 is indicative of an impedance of the body tissue within the body tissue circuit path 212 to a current flowing between the third and fourth electrodes 304, 306.

A measure of voltage drop across first and second electrodes 204, 206 together with a measure of the current waveform signal conducted through the third and fourth electrodes 308, 306 is used to determine an impedance measurement signal Z(t) across the body tissue signal path 212 according to the following relationship:

Z(t)=Δv(t)/i(t) (7)

where,

Δv(t)=v1(t)−v2(t) (8)

The parameter i(t) represents a current waveform signal conducted through a body tissue electrical path 212 between the first and second electrodes 204, 206. The parameter v1(t) represents a first voltage waveform signal v1(t) at the first electrode 204, and the parameter v1(t) represents a second voltage waveform signal v2(t) at the second electrode 206.

In operation the current injection circuit 303 injects a current waveform signal i(t) that flows via the fourth electrode 306 to the fourth tissue surface site 310, though human body tissue, which includes at least a portion of the chest cavity 214 and the heart 102, to the third tissue surface site 308, and from there to the third electrode 304. In some embodiments, the current waveform i(t) is a steady state waveform. During the flow of current i(t), the voltage signal measurement circuit 302 produces the Δv(t) representing the voltage drop across the body tissue signal path 212. Changes in the difference between v1(t) and v2(t) in the course of the flow of the current waveform i(t) are indicative of changes in the impedance of the body tissue electrical path 212 between the first and second tissue sites 208, 210, which can be caused by the occurrence of blood pulse waves ejected from the heart 102. It will be appreciated, however, that there are other causes of changes in the impedance of the body tissue that can show up in the impedance measure, such as changes due to breathing/movement of the lungs. The impedance effects of these other changes are separated out through signal processing.

In accordance with some embodiments, the voltage signal measurement circuit 302 includes a voltage difference amplifier, and the signal Δv(t) represents an amplifier output signal having a waveform that is indicative of the difference between v1(t) and v2(t). In some embodiments the, the voltage signal measurement circuit 302 can be implemented using a differential amplifier. A change in the shape and blood fluid content of the heart 102 during a heartbeat results in a change in the impedance of the body tissue circuit path 212, which in turn, results in a change in the Δv(t) waveform signal during current flow i(t). The voltage signal measurement circuit 302 monitors the voltage difference between the voltage signals on the first and second electrodes 204/304, 206/306 and produces the Δv(t) signal, which has a waveform indicative of the voltage difference, which is indicative of impedance of the body tissue circuit path 212. The beating of the heart results in changes in the impedance of the chest cavity, which causes changes in the Δv(t) waveform indicative of those changes in impedance.

Capacitor C_{ISO1} and Resistor R_{LIMIT} are electrically coupled between a node A, which is coupled to the current injection circuit 312, and the fourth electrode 306 to provide electrical protection and isolation. The voltage signal measurement circuit 302 provides a voltage difference measurement (v1(t)−v2(t)) as an output signal on a node B. A capacitor C₄ is electrically coupled between a node C, which is coupled to the current injection circuit 312, and the third electrode 304 to provide electrical protection and isolation.

The impedance voltage signal measurement circuit 302 includes a first voltage signal input node 332 electrically coupled to monitor the first electrode 204 and includes a second voltage signal input node 334 electrically coupled to motor the second electrode 206. A capacitor C₂ is electrically coupled between the first voltage signal input node 332 and the first electrode 304, and capacitor C₃ is electrically coupled between the second voltage signal input node 334 and the second electrode 206 to provide electrical protection and isolation.

In an alternative embodiment, the current waveform i(t) may be injected through the same electrodes used to measure voltage drop across the body tissue circuit path 212. However, the four electrode embodiment described above with separate electrical paths for the current injection and for impedance measurement removes some signal artifacts that may arise in an alternate two electrode implementation and generally provides more accurate results.

Still referring to FIG. 4, in accordance with some embodiments, the first and third electrodes 204, 304 are located in band mount structure 316 suited to be secured to a hand 318 and/or wrist 320, and the second, and fourth electrodes 206, 306 are located in a finger mount structure 322 suited to be secured to a finger 324 of the other hand 326. Thus, changes in impedance across the left and right hands 318, 326 can be used to measure changes in shape and blood fluid content of the chest cavity 214. In some embodiments, the hand and wrist electrodes can be located in the same band with the wrist electrodes on the bottom side of the band making contact with the hand/wrist while the finger electrodes are located on one of the outer surfaces of the band allowing the user to make contact with them when needed.

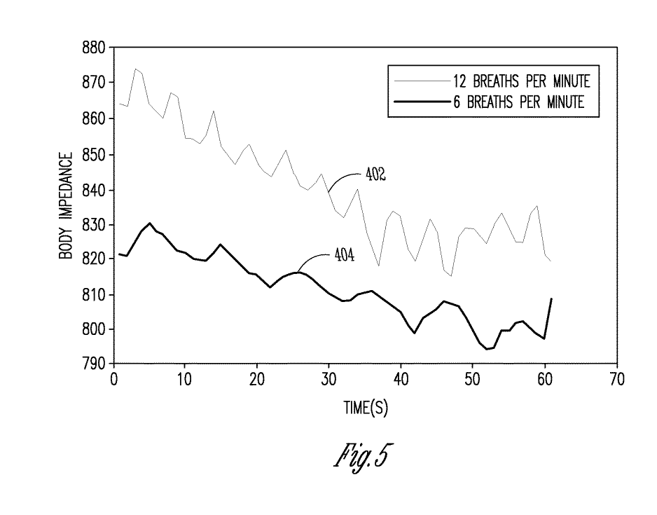

FIG. 5 is an illustrative graph showing an experimental result of impedance measurement across the chest cavity versus respiration rate for two different respiration rates. A first curve 402 indicates impedance measurements during a 60 second time frame during which the breathing rate was twelve breaths per minute. The twelve peaks in the first curve indicate a distinct change in impedance for each of the twelve breaths. The impedance rises and falls twelve times in the course of the twelve breaths in which the lung volume changes twelve times. A second curve 404 indicates impedance measurements during a 60 second time frame during which the breathing rate was six breaths per minute. The six peaks in the second curve indicate a distinct change in impedance for each of the six breaths. The impedance rises and falls six times in the course of the six breaths in which the lung volume changes six times. Thus, the example demonstrates that a change in lung volume results in a change in body tissue current path that crosses the chest cavity. The principles of measurement of impedance changes due to breaths during respiration illustrated in the experimental results of FIG. 5 are similarly applicable to measurement of impedance changes due to heartbeat.

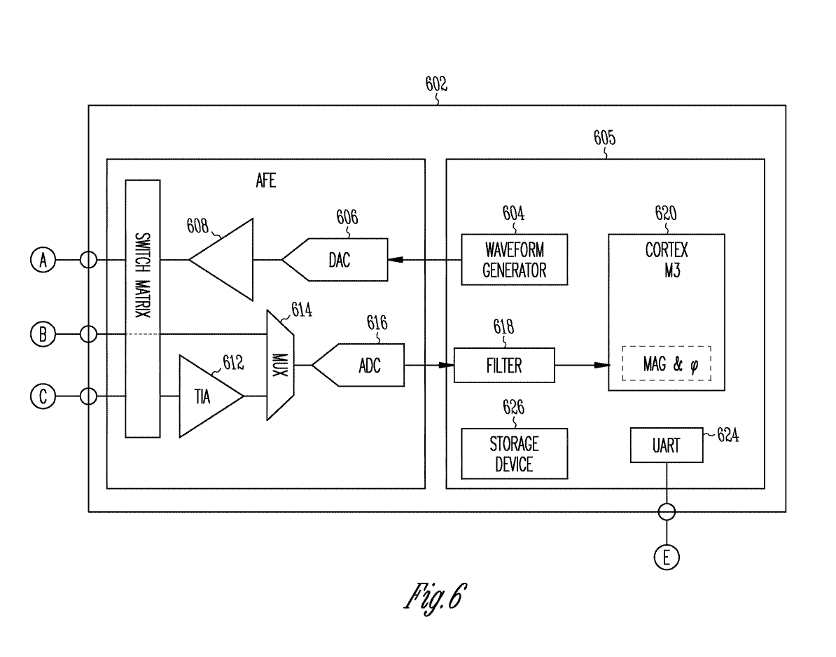

FIG. 6 is an illustrative block diagram showing certain other details of the voltage signal measurement circuit 302 in accordance with some embodiments. In particular, the signal measurement circuit 302 includes a signal processor 602 configured to perform certain functions. To implement the current injection system 312 component of the voltage measurement system 302, a waveform generator 604 produces a digital representation of the current waveform signal i(t). A DAC 606 converts the digital waveform to an analog representation. An amplifier circuit 608 provides an amplified version of the analog representation. A switch matrix 610 is electrically coupled to monitor nodes A and C. The switch matrix 610 provides the amplified version of the analog representation the signal i(t) to node A for injection to the body tissue the second electrode 306.

To compute a Z(t) waveform signal, the switch matrix 610 is electrically coupled to receive the voltage difference measurement signal Δv(t) provided on node B. The multiplexer circuit 614 selects the signal Δv(t) and provides it to ADC 616, which converts it to a digital representation. The switch matrix 610 receives at node C a return version of the signal i(t) conducted out from the body tissue via the first electrode 304. A transconductance amplifier 612 amplifies the return version of the i(t) signal received on node C. The multiplexer circuit 614 selects the return version of the current signal i(t) and provides it to ADC 616, which converts it to a digital representation.

A signal processing module 618 produces the Z(t) signal based upon the Δv(t) signal received on node B and the i(t) signal received on node C. In accordance with some embodiments, the signal frequency filter module 618 filters the received signals so as to separate signal components indicative of changes in impedance due to ejection of blood wave pulses from the heart from signal components indicative of changes in impedance due to other events. It will be appreciated that certain changes in the value of impedance Z(t) are indicative of occurrence of the heart ejecting blood pulses to peripheral regions of the circulatory system. Other changes in the value of Z(t) are indicative other events such as respiration or of other physical movements by the human subject. For example, at rest, the rate in respiration for an adult typically is at frequencies less than 0.33 Hz. At rest, the rate of heartbeat typically for an adult is greater than 0.5 Hz. Consequently, high pass frequency signal filtering can be used to separate impedance changes due to heartbeat from impedance changes due to respiration. In some embodiments, the signal frequency filter module 618 uses a Discrete Fourier Transform (DFT) algorithm to filter the received signals. Thus, the signal processing module 618 produces a blood pulse wave ejection signal ΔZ_{H}(t), also referred to herein as a first signal, that is indicative of times at which a change in impedance of the chest cavity indicates an occurrence of ejection of a blood pulse to the circulatory system. The cortex M3 module 620 is used to control the different blocks of the sensor, perform low level digital signal processing, and communicate the data to other devices through the Universal Asynchronous Receiver Transmitter (UART) interface 624. In addition, the Cortex M3 optionally receives commands from other devices (not shown) through interface 624 as well. The cortex M3 can be replaced with higher end processors if more on-chip processing is required. A storage device 626, such as DRAM, ROM, Flash or Disc, is included to store computer readable instructions and/or data to configure the processor 602.

An I/O interface at node E is coupled to a UART interface 624 to send and receive signals to and from an outside source. In accordance with some embodiments the blood pulse wave ejection signal is provided as an output signal at node E.

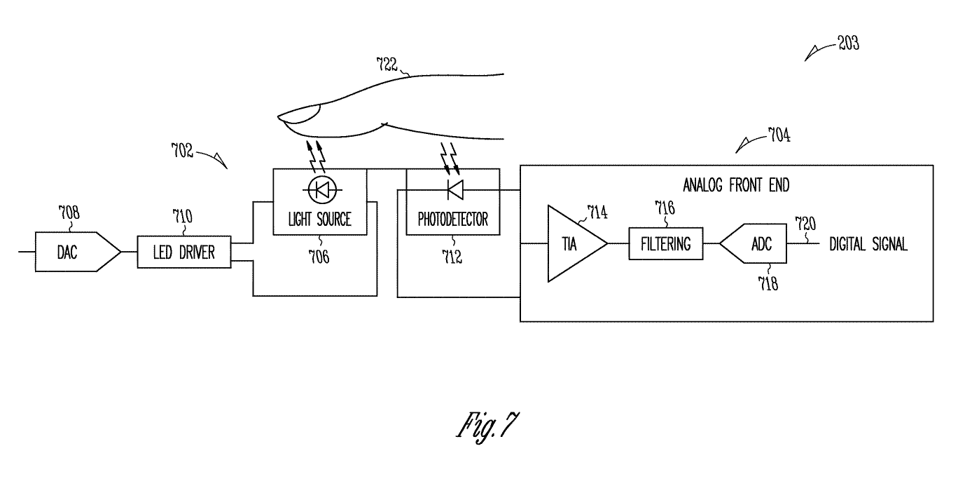

FIG. 7 is an illustrative schematic block diagram showing certain details of the blood pulse detection sensor 203 of FIG. 2, in accordance with some embodiments. The sensor 203 includes a light source 702 and a photodetector 704. The light source 702 includes a light emitting diode (LED) 706. A DAC circuit 708 provides a signal to an LED driver circuit 710, which drives the LED 706. The photodetector 704 includes a photodetector diode 712. An amplifier circuit 714 amplifies a signal produced by the photodetector 704. A filtering circuit 716 filters the amplified signal. An ADC circuit 718 converts the amplified and filtered representation of the photodetector output signal from an analog to a digital representation and provides the digital version of the photodetector output signal, also referred to herein as a second signal, on line 720.

In operation, the light source 706 and the photodetector 712 are placed in close proximity to a body site peripheral to the heart at which the arrival times of blood pulses are to be detected. The example show in the drawing shows them disposed close to a finger 722, although they can be placed in close proximity to other body sites such as wrist, ear lobe, ear cavity, etc. More particularly, in accordance with some embodiments, the light source 706 and the photodetector 712 are position relative to a body site so as to be suitable for photoplethysmography (PPG) measurements in which the output signal on line 720 is indicative of local blood volume changes ΔVᵢ that occur upon the arrival of a blood pulse wave at a body site where the optical sensor is sensing. A PPG sensor detects blood pressure change by detecting a change in the reflectivity of light through the skin surface, which is indicative of the arrival of a blood pulse wave.

Thus, it will be understood that the blood pulse detection sensor 203 detects arrival of a blood pulse through detection of changes in blood dynamics such as changes in blood density, changes in blood pressure or changes blood flow, for example. As explained above, in some embodiments the blood pulse detection sensor 203 includes a PPG sensor that detects arrival of a blood pulse based upon a change in blood volume. In alternative embodiments, the blood pulse detection sensor 203 can be implemented to detect arrival of a blood pulse based upon a change in blood pressure or to detect arrival of a blood pulse based upon a change in blood flow, for example.

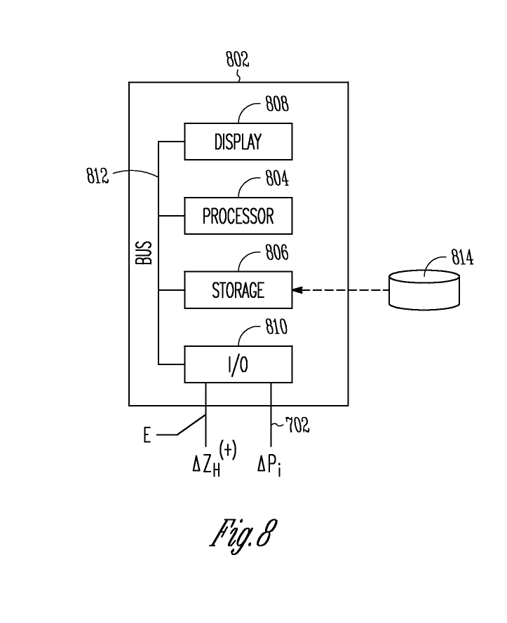

FIG. 8 is an illustrative drawing showing a computer system 802 coupled to process PTT related information in accordance with some embodiments. The computer system 802 includes a processor 804, non-transitory storage 806, such as disc, RAM, ROM disc and/or Flash, a graphic display monitor 808, I/O 810 and a bus circuit 812 providing a communication path among the components. The computer system 802 is electrically coupled to receive impedance change signal information via node E of the signal processor 602 and is coupled to receive blood pressure change signal information via output line 702 of the blood pulse detection sensor 203. Both node E and the output line 702 can be wired or wireless. The computer system 802 is configured to compute PTT information by determining a time difference between times of occurrence of a change in impedance signal value indicative of a blood pulse ejected from the heart left ventricle to the aorta, detected using the impedance measurement circuit 202/302, and time of arrival of that same blood pulse, detected using the blood pulse detection sensor 203, at a location peripheral to the heart. More particularly, the computer system 802 is coupled to receive the blood pulse wave ejection signal ΔZ_{H}(t) provided on node E and to receive the blood pressure change signal ΔPᵢ on line 720. The storage device 806 includes computer program code to configure the computer system to determine PTT based upon impedance change measurements and peripheral site blood pressure change measurements.

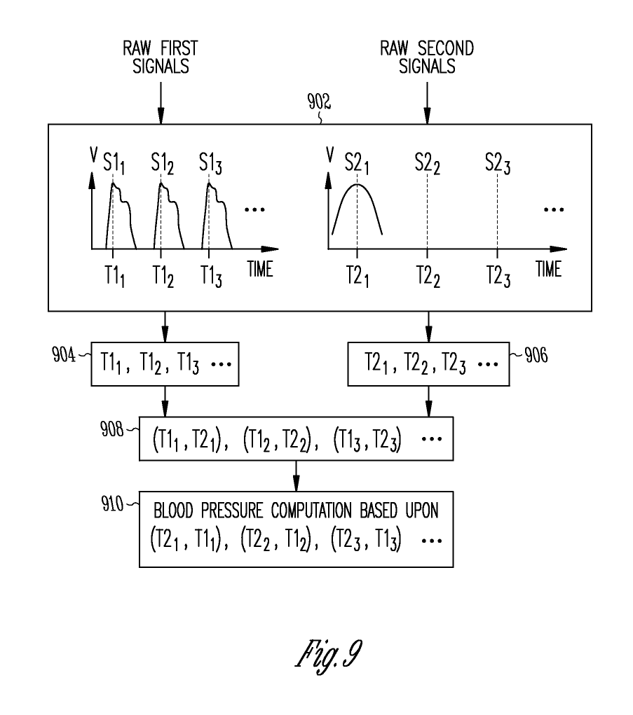

FIG. 9 is an illustrative flow diagram representing configuration of the processor 804 of the computer system of FIG. 8 to determine PTT values in accordance with some embodiments. Computer readable computer program code that includes instructions stored in a storage device 806 can be used to configure the processor to determine PTT values. Module 902 configures the processor to receive from the impedance sensor circuit 202 of FIG. 4, raw first signals S1₁, S1₂, S1₃, etc., indicative times of occurrences of changes of impedance and receives from the blood pulse detection sensor 203 of FIG. 7, raw second signals S2₁, S2₂, S2₃, etc., indicative of change changes in blood pressure. Module 902 filters and conditions the received impedance change signal and blood pressure change signals to remove interferers and artifacts (such as motion) are removed from the sensors raw data. Module 904 configures the processor to determine a sequence of impedance change time reference signals T1₁, T1₂, T1₃, etc., that indicate each time of occurrence of a first signal. It will be appreciated that a sequence of occurrences of a blood pulse wave ejections results in the sequence of first signal occurrences S1₁, S1₂, S1₃, etc., corresponding to the ejections. Thus, a first sequence of reference time signals T1₁, T1₂, T1₃, etc., is produced that corresponds to the sequence of first signal occurrences S1₁, S1₂, S1₃, etc. Module 906 configures the processor to determine a sequence of blood pressure change time reference signals T2₁, T2₂, T2₃, etc., that indicate each time of occurrence of a second signal. It will be appreciated that each occurrence of a blood pulse wave ejection wave results in an occurrence (after a PTT delay) of a second signal corresponding to occurrence of an arrival of that pulse wave at a body location monitored by the blood pulse detection sensor 203. Thus, a second sequence occurrence reference time signals T2₁, T2₂, T2₃, etc., is produced that corresponds to the sequence of second signal occurrences S2₁, S2₂, S2₃, etc. For each of the first and second signals, reference times can be determined based upon a local maximum, local minimum, zero crossing, or other defined feature of a measured impedance change waveform or pressure change waveform to determine its time of occurrence. The fourth module 908 configures the processor to associate first and second time reference signals that correspond to the same cardiac cycle, e.g., (T1₁, T2₁), (T1₂, T2₂), (T1₃, T2₃), etc. Module 910 configures the processor to use the associated first and second signal time of occurrence reference points to compute a time delay between them (PTT), e.g. (T2₁−T1₁), (T2₂−T1₂), (T2₃−T1₃), etc., and to convert the time delay to a blood pressure measurement. In accordance with some embodiments, one or more of relationships expressed in formulas (1)-(6), or other suitable formulations, can be used to convert PTT to a blood pressure determination or to a blood pressure change determination.

In accordance with some embodiments, a non-transitory storage device 808 external to the computer system 802, can be used to deliver the computer program code. The external storage device 808 may include a thumb drive or may include remote storage accessible over a network (not shown) such as the internet, for example.

The foregoing description and drawings of embodiments in accordance with the present invention are merely illustrative of the principles of the invention. For example, the storage device 626 can store computer readable code instructions to configure processor 602 to perform the process of FIG. 9. Also, for example, a ballistocardiogram (BCG) measurement can be used instead of an impedance measurement to detect the time of occurrence of ejection of a blood pulse. As another alternative, an electrocardiogram (ECG) measurement can be used instead of an impedance measurement to detect the time of occurrence of ejection of a blood pulse. Moreover, an impedance plethysmography (IPG) sensor can be used instead of a PPG sensor. Therefore, it will be understood that various modifications can be made to the embodiments by those skilled in the art without departing from the spirit and scope of the invention, which is defined in the appended claims.

1. A blood pressure monitoring system comprising:
an impedance measurement circuit configured to detect a change in impedance indicative of an occurrence of a blood pulse wave ejection from the heart;
a blood pulse detection circuit configured to detect arrival of the blood pulse wave at a body site peripheral to the heart; and
a non-transitory computer readable storage device that includes computer program code to configure a processor to compute pulse transit time (PTT) based at least in part upon a difference in a time of occurrence of the blood pulse wave ejection from the heart first signal and a time of occurrence of the arrival of the blood pulse wave at a body site peripheral to the heart.

2. The system of claim 1 further including:
a first electrode;
a second electrode;
a third electrode;
a fourth electrode;
a current injection circuit; and
a voltage difference measurement circuit,
wherein the current waveform generator is coupled to inject a current between the first and second electrodes; and
wherein the voltage difference measurement circuit is coupled to monitor a voltage difference between the third and fourth electrodes.

3. The system of claim 2,
wherein the first and third electrodes are secured to a first mount structure suited for mounting to a first anatomical location; and
wherein the second and fourth electrodes are secured to a second mount structure suited for contact to a second anatomical location.

4. The system of claim 2,
wherein the current injection circuit includes a current waveform generator.

5. The system of claim 1 further including:
a first electrode;
a second electrode;
a current injection circuit; and
a voltage difference measurement circuit,
wherein the current injection circuit is coupled to inject a current between the first and second electrodes; and
wherein the voltage difference measurement circuit is coupled to monitor a voltage difference between the first second electrodes.

6. The system of claim 5,
wherein the first electrode is secured to a first mount structure suited for mounting to a first anatomical location; and
wherein the second electrode is secured to a second mount structure suited for mounting to a second anatomical location;

7. The system of claim 5,
wherein the current injection circuit includes a current waveform generator.

8. The system of claim 1 further including;
a frequency filter circuit configured to identify signal components within a voltage difference measurement signal produced using the voltage difference measurement circuit that are indicative of changes in impedance due to ejection of blood wave pulses from the heart.

9. The system of claim 1,
wherein the blood pressure measurement circuit includes a light source and a photodetector.

10. The blood pressure monitoring system of claim 1,
wherein the impedance measurement circuit is configured to produce a first signal indicative of a change in impedance indicative of an occurrence of a blood pulse wave ejection from the heart; and
wherein the blood pulse detection circuit is configured to produce a second signal indicative of an occurrence of an arrival of the blood pulse wave at a body site peripheral to the heart; and
wherein the non-transitory computer readable storage device that includes computer program code to configure a processor to compute pulse transit time (PTT) based at least in part upon a difference in a time of occurrence of the first signal and a time of occurrence of the second signal.

11. The system of claim 10,
wherein the computer program code includes code to configure a processor to determine a reference time of occurrence associated with the first signal and to determine a reference time of occurrence associated with the second signal and to compute a pulse transit time (PTT) based at least in part upon a difference in the determined reference time of occurrence associated with the first signal and the determined reference time of occurrence associated with the second signal.

12. The system of claim 10,
wherein the impedance measurement circuit is configured to produce a sequence of first signals indicative of a sequence of changes in impedance indicative of each occurrence of a blood pulse wave ejection from the heart for a sequence of cardiac cycles; and
wherein the blood pressure measurement circuit is configured to produce a sequence of second signals indicative of a sequence of occurrences of changes in blood pressure upon arrival of the blood pulse wave at a body site peripheral to the heart for the sequence of cardiac cycles.

13. The system of claim 10,
wherein the impedance measurement circuit is configured to produce a sequence of first signals indicative of a sequence of changes in impedance indicative of each occurrence of a blood pulse wave ejection from the heart for a sequence of cardiac cycles;
wherein the blood pressure measurement circuit is configured to produce a sequence of second signals indicative of a sequence of occurrences of changes in blood pressure upon arrival of the blood pulse wave at a body site peripheral to the heart for the sequence of cardiac cycles; and
wherein the computer program code includes code to configure a processor to determine a sequence of reference times of occurrence associated with the sequence of first signals and to determine a sequence of reference times of occurrence associated with the sequence of second signals and to compute a sequence of pulse transit times (PTT) based at least in part upon differences in the determined reference times of occurrence associated with corresponding first and second signals.

14. The system of claim 10,
wherein the impedance measurement circuit is configured to produce a sequence of first signals indicative of a sequence of changes in impedance indicative of each occurrence of a blood pulse wave ejection from the heart for a sequence of cardiac cycles;
wherein the blood pressure measurement circuit is configured to produce a sequence of second signals indicative of a sequence of occurrences of changes in blood pressure upon arrival of the blood pulse wave at a body site peripheral to the heart for the sequence of cardiac cycles; and
wherein the computer program code includes code to configure a processor to determine a sequence of reference times of occurrence associated with the sequence of first signals and to determine a sequence of reference times of occurrence associated with the sequence of second signals and to associate, with each other, determined reference times of occurrence that correspond to the same cardiac cycle and to compute a sequence of pulse transit times (PTT) based at least in part upon differences in the determined reference times of occurrence associated with corresponding first and second signals.

15. A blood pressure monitoring system including:
an impedance measurement circuit configured to detect a change in impedance indicative of an occurrence of a blood pulse wave ejection from the heart;
a blood pressure measurement circuit configured to detect an occurrence of a change in blood dynamics upon arrival of the blood pulse wave at a body site peripheral to the heart; and
a processor configured to compute pulse transit time (PTT) based at least in part upon a difference in a time of occurrence of the blood pulse wave ejection from the heart first signal and a time of occurrence of the arrival of the blood pulse wave at a body site peripheral to the heart.

16. A method to monitor blood pressure comprising:
monitoring electrical impedance at first and second body skin tissue sites;
monitoring blood pressure at a third skin tissue site; and
determining a pulse transit time (PTT) based at least in part upon a difference in a time of occurrence of a monitored change in impedance at the first skin tissue site and a monitored change in blood pressure at the second skin tissue site.

17. The method of claim 16,
wherein the first and second skin tissue sites are disposed such that the heart is located between them.

18. The method of claim 16,
wherein the first and second skin tissue sites are disposed such that a blood pulse wave ejection from the heart produces a change in impedance of a body tissue electrical path between the first and second skin sites.

19. The method of claim 16,
wherein monitoring electrical impedance at first and second body skin tissue sites includes monitoring a voltage difference between the first and second body skin tissue sites.

20. The method of claim 16,
wherein monitoring blood pressure at the third skin tissue site includes monitoring blood volume at the third skin site.

21. The method of claim 16,
wherein monitoring electrical impedance at the first and second body skin tissue sites includes producing a voltage difference measurement signal indicative of a voltage difference between voltage at the first skin tissue site and a voltage at the second skin tissue site; and further including:
filtering the voltage difference signal to identify signal components within the voltage difference measurement signal that are indicative of changes in impedance due to ejection of blood wave pulses from the heart.

22. The method of claim 16,
wherein monitoring electrical impedance at the first and second body skin tissue sites includes producing a sequence of first signals indicative of a sequence of changes in impedance indicative of each occurrence of a blood pulse wave ejection from the heart for a sequence of cardiac cycles;
wherein monitoring blood pressure at a third skin tissue site includes producing a sequence of second signals indicative of a sequence of occurrences of changes in blood pressure upon arrival of the blood pulse wave at a body site peripheral to the heart for the sequence of cardiac cycles; and
wherein determining a PTT includes determining a sequence of reference times of occurrence associated with the sequence of first signals and determining a sequence of reference times of occurrence associated with the sequence of second signals and computing a sequence of PTTs based at least in part upon differences in the determined reference times of occurrence associated with corresponding first and second signals.
